(19) United States
(12) Patent Application Publication
(10) Pub. No.: US 2006/0259101 A1
(43) Pub. Date: Nov. 16, 2006

(54) UV light irradiation machine for veterinary use

(76) Inventor: Thomas Perez, Chicago, IL (US)

Correspondence Address: Orum & Roth LLC, 53 West Jackson Blvd., Chicago, IL, 60604, US

(21) Appl. No.: 11/125,789

(22) Filed: May 10, 2005

(51) Int. Cl. A61N 5/06

(52) U.S. Cl. 607/88; 606/2

(57) ABSTRACT

Ultraviolet light is applied to an animals blood while that blood remains in the body. The UV light is applied sublingually or directly to the blood. One of more light guides with fused silica fiber optic bundles is inserted under the animal's tongue. A fiber optic strand or bundle runs through the tube to irradiate the mucus membrane and provide the UV light to the blood.

### BACKGROUND OF THE INVENTION

1. Field of the Invention

The present invention relates to a method and apparatus for sublingual application of UV light to animals.

2. Background of the Prior Art

Ultraviolet (UV) light can be used to treat a multitude of medical problems in humans and animals, including for example bacterial, viral and fungal infections, poisoning, fatigue, allergies and asthma, rheumatic diseases and arthritis, diabetes, hepatitis, and cancer. UV light sterilizes the blood and acts as an antibiotic.

The UV light is applied either to the patient's skin or directly to the blood. If the UV light is applied to the skin it is typically provided to the patient's skin either with a wrap or lamp. For example, UV light is commonly used to treat jaundiced babies. Because infant's skin is thin and the blood vessels are close to the surface, UV light is typical applied to the skin when treating jaundiced babies. A wrap of lamp is generally unsuitable for treating animals because animal skin is often thick and/or coated with fur and/or there is a fat layer under the skin. Thus, the capillaries are often not close to the surface.

Applying the UV light directly to a patient's blood supply is known as photoluminescence or UV blood illumination (UBI). UV blood illumination increases oxygen, destroys toxins and boosts the immune system. In prior art UBI for humans, a small amount of blood is drawn from the patient, up to about 250 cc. The blood that is drawn travels through a cuvette or glass chamber. The blood is repeatedly illuminated with UV light and then returned to the body. The process is repeated, typically a day or several days later. These treatments are time consuming, and require regular trips to a medical facility. In addition, trained personal must be available to provide the treatments.

There is a need for a method of providing UV light to animals. There is a need for system which would allow the animal's blood stream to be illuminated, not just a small portion of it as occurs with traditional UBI. There is a need for a system that is convenient for the animal and caregiver. There is a need for a system which allows the animal to be treated when it is located without the need to move it to a veterinary or other medical center. There is a need for a system which can be administered by the owner, technician, veterinarian, or animal caregiver without requiring that the veterinarian or veterinarian assistant always be present. There is a need for a simple system that can be used by the patient in his home.

There is a need for a system that allows for round the clock treatments or other regular treatments such as pulsed treatment or automatic periodic treatments.

There is a need for a blood illuminator that reduces the risk of infection from removing blood. There is a need for a system that reduces the number of needle sticks the animal must endure.

There is a need for a system that allows the blood to be treated on an as needed basis, such as based on the animals current symptoms at a particular time or response to the treatment.

### SUMMARY OF THE INVENTION

The present invention is a UV device that irradiates the mouth under the tongue or which is implanted under the animal's skin. The UV pack has a battery or other power supply and a UV light source. The UV pack or device is inserted into the patient's mouth, preferably under the tongue. Fiber optic strand(s) run through one or more tube's to illuminate the capillaries under the tongue or to directly illuminate the blood for a implantable device.

U.S. application Ser. No. 10/926,209 filed Aug. 25, 2004, No. 11/088,613 filed Mar. 23, 2005, and No. 11/076,169 filed Mar. 9, 2005 are hereby incorporated in their entirety.

### BRIEF DESCRIPTION OF THE DRAWINGS

FIG. 1 is an implantable UV light irradiation delivery device;

FIG. 2 is a sub-lingual UV light irradiation delivery device; and

FIG. 3 is a sub-lingual UV light irradiation delivery device.

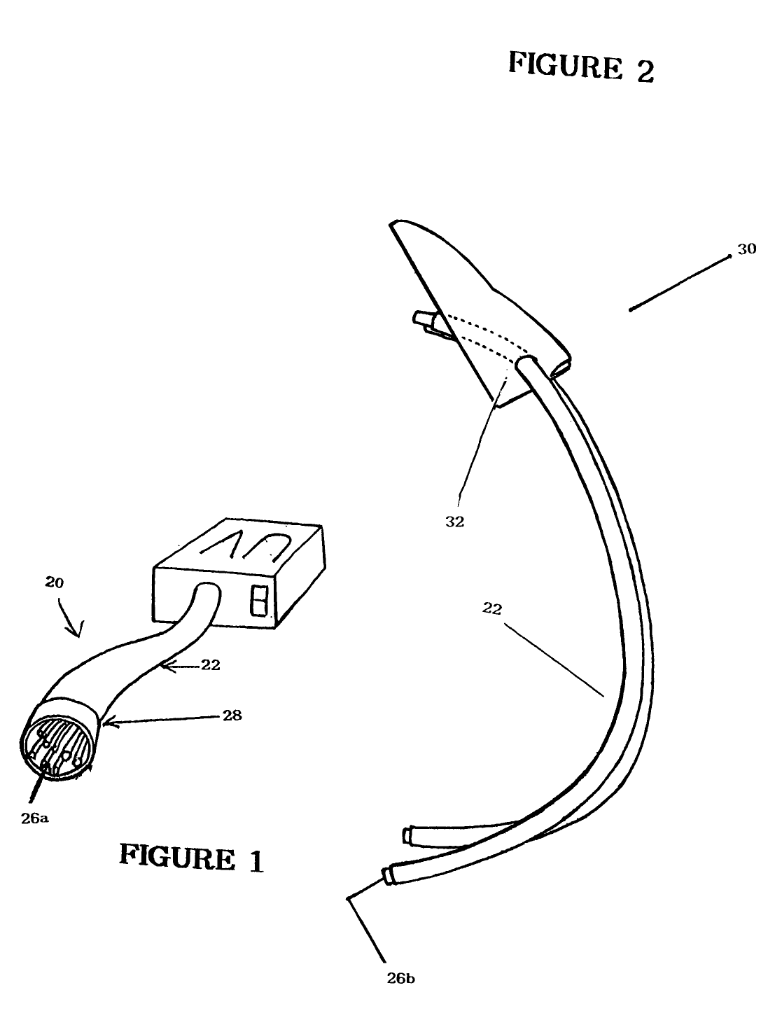

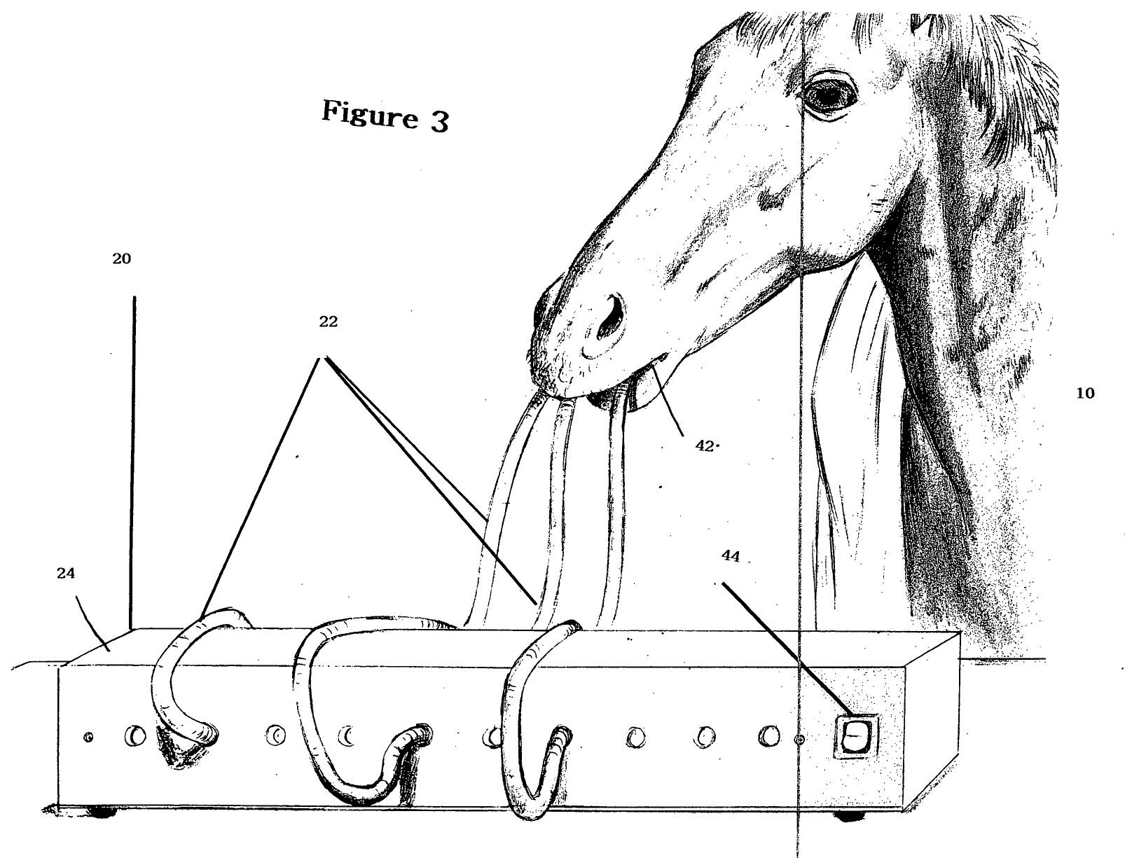

### DETAILED DESCRIPTION OF THE INVENTION

Ultraviolet light (UV) can is used to treat many diseases including bacterial, viral and fungal infections, poisoning, fatigue, allergies, hepatitis, cancer and HIV. UV light increases the oxygen combining power of the blood, destroys toxins, viruses, fungi, bacteria, and boosts the immune system. UV light also sterilizes the blood and acts as an antibiotic. Preferably, the UV light is selected based on the condition being treated. For some treatments UV-C is preferred, for others UV-A is preferred. Other conditions may be best treated with UV-B or a combination of various UV wavelengths. Animals get many of the same diseases as humans as well as other diseases which need treatment. For example, horses or newborn colts may develop a bacterial infection for which the current treatment is very high doses of antibiotic. The horse is likely to die from the treatments. Cats gets feline leukemia, feline HIV, and feline diabetes.

The invention is a UV device 20 that is connectable to an animal 10 by being inserted under the tongue. The capillaries under the tongue are close to the surface. These capillaries are very sensitive. Capillary exposure of the mucus membrane is significantly greater than other exposed body surfaces. The greater capillary exposure allows for greater penetration of the ultraviolet spectrum.

Alternatively, UV device 20 could be surgically implanted under the animal's skin, a UV light guide 22 would then be inserted into a vein or artery to directly illuminate the blood.

UV device 20 comprises housing 24, battery, power cable connectable to an outlet or generator, or other power source (not shown) and UV light source. The UV light source may be in the housing or it may be placed at the point of illumination. Preferably, UV light source is LEDs emitting UV light at a therapeutic wavelength. Alternatively, a plurality of LEDs, having one or more different wavelengths of UV light could be used.

In one embodiment, UV device 20 has at least one light guide 22 made of rubber, plastic or other material for housing one or more fiber optic strands 26a. The light guide may be made of a flexible, rigid or semi-rigid material. The material used may be selected based on the species of animal being treated, their propensity to damage or play with the light guide, the conditions under which the animal is being treated or based on other considerations. Fused silica fiber optic bundles 26b are preferred because they do not emit any heat. The fiber optic bundles 26b preferably deliver UV light sublingually. In yet another alternative, UV light source is a LED or small light bulb at the end of the flexible tube adapted to directly irradiate the mucus membrane under the tongue. Alternatively, a liquid core light guide or other known light guide can be used. One end of the light guide is connectable to the housing and the other end is placed when the irradiation is desired. Emitted UV radiation travels to the end tip of the light guide 22 and is emitted. Emitted radiation directly illuminates patient's blood or the area under the tongue. Sublingual administration of the UV radiation is preferred because the capillaries are close to the surface on the mucus membrane under the tongue. The UV light is thus able to treat the blood more effectively. Optionally, the end of the light guide has a mouthpiece 30. The mouthpiece can serve to direct the light to the underside of the tongue. A mouthpiece 30 that has been formed to the shape of the animal's mouth 42 can make the treatment more comfortable for the animal 10.

UV light is administered under the tongue. The optional mouthpiece 30 is preferably adapted to hold and align the light source or light guide 22 under the tongue. Mouthpiece 30 is inserted into animal's mouth under the tongue. In one embodiment, the mouthpiece 30 has at least one aperture 32 through which the light guide 22 is inserted. The tubing may be adjustably inserted through the at least one aperture 144 to allow for individual adjustment by the patient 10 or doctor to a preferred treatment location under the patient's tongue. Alternatively, tube 22 can be mounted in the preferred position such that each time the mouthpiece 30 is used, the light is administered at the same location. The mouthpiece 30 may be molded to the shape of the individual animals' 10 mouth, molded based on animal species and size or standardized mouthpieces can be used. The mouthpiece can optionally be a light guide when the underside of the mouthpiece would emit light in the therapeutic wavelength.

In an alternative embodiment, the light guide or light guides 22 are manually placed in the desired position and optionally secured. For example for a horse, the light guide may be secured under the tongue in the same manner that a tongue depressor is secured.

It is preferable if the light guide 22 is inserted into the animal's mouth 42 in an area where it will cause the least discomfort to the animal and where the animal is least likely to damage the light guide. For example, for a horse, the light guide 22 may be inserted on the side of the mouth rather than in the front. The number of light guides 22, intensity, therapeutic wavelegth and/or treatment times of the light is determined by the size of the animal 10, species, age of the animal, condition being treated and/or based on other variables.

In another embodiment, the light guide or light guides 22 are inserted through the animal's nostril and irradiate the mucus membrane of the throat, stomach or other membrane with capillaries close to the surface.

In another embodiment, UV device 20 is surgically attached to animal 10 or implanted in the animal 10. Light guide 22 is a flexible catheter and has transparent cover 28 at the tip end. In an alternative embodiment, light source such as a LED, or miniature light bulb located at the tip or distal end of the light guide of a UV device implanted in the animal directly illuminates the blood.

The UV device 20 allows UV light to be supplied directly to the blood or to irradiate the blood supply very close to the surface. Thus, large amounts of blood or even the entire blood supply of an animal can be treated.

The animal can be connect to UV device 20 when a treatment is needed. Alternatively, UV device can be implanted into the animal and be turned on only for treatment. UV device 20 could be turned on and off manually with a switch 44 or by another know method. Alternatively, UV device 20 could automatically turn off the light source after a set treatment time, such as fifteen minutes. UV device 20 could have a controller such as a computer or other smart interface that limits the number of treatments given time period, limits the total amount of treatment time in a given time period, automatically provides treatments, pulses the light source, or provides only particular wavelengths. The computer or other smart interface could keep a treatment record. The computer or other smart interface could communicate wirelessly, via the Internet or through other electronic means to automatically update the treatment records for that animal. The computer preferably can automatically adjust treatment time, wavelength or other variables based on symptom.

I claim:

1. A UV blood illuminator comprising:
a housing;
a power supply;
a UV light source powered by said power supply; said UV light source in said housing and
at least one light guide, each said light guide having a first end connected to a UV light source and a second end adapted for connection with an animal.

2. The UV blood illuminator of claim 1 further comprising at least one fiber optic strand in said light guide.

3. The UV blood illuminator of claim 1 wherein the UV light source comprises at least one LED.

4. The UV blood illuminator of claim 1 wherein the UV light source is a medical grade UV light bulb.

5. The UV blood illuminator of claim 1 further comprising a mouthpiece adapted to be inserted under a patient's tongue.

6. The UV blood illuminator of claim 5 wherein the mouthpiece is adapted to secure the light guide under the animal's tongue.

7. The UV blood illuminator of claim 5 wherein the mouthpieces adapted to direct the irradiation from the light guide.

8. The UV blood illuminator of claim 7 wherein the mouthpiece a light guide.

9. The UV blood illuminator of claim 1 wherein the second end portion of the tube is transparent.

10. The UV blood illuminator the claim 1 wherein the at least one light guide is adapted to be inserted in the animals nostril.

11. The UV blood illuminator of claim 2 wherein the fiber optic strand is a fused silica fiber bundle.

12. The blood illuminator of claim 1 further comprising a controller.

13. The blood illuminator of claim 12 wherein the controller comprises an on/off switch.

14. The blood illuminator of claim 12 wherein the controller automatically controls the light source.

15. The blood illuminator of claim 14 wherein the controller automatically controls the light source by pulsing the light, by automatically shutting off the light after a specified period of time, by automatically activating the light source at a specified time or by combinations thereof.

16. The UV blood illuminator of claim 12 wherein the controller is a computer.

17. The UV blood illuminator of claim 16 wherein the computer is adapted to maintain and/or transmit treatment records.

18. The blood illuminator of claim 1 wherein the blood illuminator is adapted to be implanted in the animal and the second end directly illuminates blood in an artery or vein.

19. A method of illuminating blood comprising:
directing UV irradiation to an animals' mucus membrane.
illuminating the mucus membrane for a period of time with UV light.

20. The method of claim 19 further comprising the steps of securing at least one light guide in the animal's mouth, under the animal's tongue.
